(19) United States
(12) Patent Application Publication
(10) Pub. No.: US 2018/0240546 A1
(43) Pub. Date: Aug. 23, 2018

(54) REGULATORY AND PROCEDURAL FRAMEWORK COMPLIANCE AND HOSPITAL STAFF COMMUNICATION AND DEVELOPMENT SYSTEM AND PROCESSES FOR FACILITATING HOSPITAL STAFF COMMUNICATION, DEVELOPMENT, AND COMPLIANCE WITH REGULATORY AND PROCEDURAL FRAMEWORKS

(71) Applicant: Margaret Christine Pfeiffer, Long Beach, CA (US)

(72) Inventor: Margaret Christine Pfeiffer, Long Beach, CA (US)

(21) Appl. No.: 15/901,021

(22) Filed: Feb. 21, 2018

Related U.S. Application Data
(60) Provisional application No. 62/462,197, filed on Feb. 22, 2017.

(51) Int. Cl. G16H 40/20; H04L 29/06

(52) U.S. Cl. CPC G16H 40/20; H04L 63/08

(57) ABSTRACT

A regulatory and procedural framework compliance and hospital staff communication and development system and processes for facilitating hospital staff communication, development, and compliance with regulatory and procedural frameworks are disclosed. In some embodiments, the system and processes facilitate real-time, secured communication available on mobile devices that provide access to communications at the convenience of the staff who need to receive such communications, and who need to be able to perform required education modules on clinical subjects. By using the system and the processes to disseminate and receive vital clinical information, both managers and staff can have real-time access in any location at any time of day or night that is convenient for the employees.

This application claims benefit to U.S. Provisional Patent Application 62/462,197, entitled “SYSTEM AND PROCESS FOR FACILITATING HOSPITAL STAFF COMMUNICATION, DEVELOPMENT, AND COMPLIANCE WITH REGULATORY AND PROCEDURAL FRAMEWORKS,” filed Feb. 22, 2017. The U.S. Provisional Patent Application 62/462,197 is incorporated herein by reference.

### BACKGROUND

Embodiments of the invention described in this specification relate generally to communication systems, and more particularly, to a regulatory and procedural framework compliance and hospital staff communication and development system and processes for facilitating hospital staff communication, development, and compliance with regulatory and procedural frameworks.

Communication to staff in clinical departments of hospitals is difficult because of varied and irregular hours staff work. Ensuring clinical staff is all aware of new protocols, quality results and changed policies is extremely difficult for department directors who manage several full time employees (FTEs) who work night shift, or weekends only.

Currently there is no other application program for facilitating real-time, secured communication available on mobile devices.

Therefore, what is needed is a way to facilitate real-time, secured communication available on mobile devices which provides access to communications at the convenience of the staff who need to receive such communications, and who need to be able to perform required education modules on clinical subjects.

### BRIEF DESCRIPTION

A novel regulatory and procedural framework compliance and hospital staff communication and development system and processes for facilitating hospital staff communication, development, and compliance with regulatory and procedural frameworks are disclosed. In some embodiments, the system and processes facilitate real-time, secured communication available on mobile devices that provide access to communications at the convenience of the staff who need to receive such communications, and who need to be able to perform required education modules on clinical subjects. By using the system and the processes to disseminate and receive vital clinical information, both managers and staff can have real-time access in any location at any time of day or night that is convenient for the employees.

In some embodiments, a process for facilitating hospital staff communication, development, and compliance with regulatory and procedural frameworks includes (i) receiving, from a manager computing device, information provided by a manager and related to a hospital department for viewing by staff associated with the hospital department, (ii) posting the information related to the hospital department on a newsfeed for the hospital department that is accessible to the manager via the manager computing device and the staff via a mobile app that runs on a personal mobile device for each staff member of the staff, (iii) receiving, from a first mobile device of a first staff member, a set of user credentials for a first staff member login, (iv) authenticating the set of user credentials for the first staff member login, (v) providing, to the first mobile device of the first staff member, secured access to the newsfeed when the set of user credentials for the first staff member login are successfully authenticated, and (vi) transmitting the posted information related to the hospital department to the first mobile device for display on a screen of the first mobile device when the first mobile device securely accesses the newsfeed and the first staff member selects a newsfeed entry in the newsfeed associated with the posted information.

The preceding Summary is intended to serve as a brief introduction to some embodiments of the invention. It is not meant to be an introduction or overview of all inventive subject matter disclosed in this specification. The Detailed Description that follows and the Drawings that are referred to in the Detailed Description will further describe the embodiments described in the Summary as well as other embodiments. Accordingly, to understand all the embodiments described by this document, a full review of the Summary, Detailed Description, and Drawings is needed. Moreover, the claimed subject matters are not to be limited by the illustrative details in the Summary, Detailed Description, and Drawings, but rather are to be defined by the appended claims, because the claimed subject matter can be embodied in other specific forms without departing from the spirit of the subject matter.

### BRIEF DESCRIPTION OF THE DRAWINGS

Having thus described the invention in general terms, reference is now made to the accompanying drawings, which are not necessarily drawn to scale, and which show different views of different example embodiments, and wherein:

FIG. 1 conceptually illustrates a block diagram of a mobile messaging board app for healthcare employees in some embodiments.

FIG. 2 conceptually illustrates a process in some embodiments for facilitating hospital staff communication, development, and compliance with regulatory and procedural frameworks by way of computer systems.

FIG. 3 conceptually illustrates a systems administrator process for facilitating hospital staff communication, development, and compliance with regulatory and procedural frameworks in some embodiments.

FIG. 4 conceptually illustrates a manager reports and notes process for facilitating hospital staff communication, development, and compliance with regulatory and procedural frameworks in some embodiments.

FIG. 5 conceptually illustrates a manager accesses and communications process for facilitating hospital staff communication, development, and compliance with regulatory and procedural frameworks in some embodiments.

FIG. 6 conceptually illustrates an employee (end user) sign up interface process in some embodiments.

FIG. 7 conceptually illustrates an employee (end user) interactions with management process in some embodiments.

FIG. 8 conceptually illustrates a network architecture of a cloud-based regulatory and procedural framework compliance and hospital staff communication and development system in some embodiments.

FIG. 9 conceptually illustrates an electronic system with which some embodiments of the invention are implemented.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

Some embodiments include a novel regulatory and procedural framework compliance and hospital staff communication and development system and novel processes for facilitating hospital staff communication, development, and compliance with regulatory and procedural frameworks. In some embodiments, the system and processes facilitate real-time, secured communication available on mobile devices that provide access to communications at the convenience of the staff who need to receive such communications, and who need to be able to perform required education modules on clinical subjects. By using the system and the processes to disseminate and receive vital clinical information, both managers and staff can have real-time access in any location at any time of day or night that is convenient for the employees.

As stated above, communication to hospital staff is difficult because of varied and irregular hours staff work. Ensuring clinical staff is all aware of new protocols, quality results and changed policies is extremely difficult for department directors who manage several FTEs who work night shift, or weekends only. Embodiments of the regulatory and procedural framework compliance and hospital staff communication and development system and the processes for facilitating hospital staff communication, development, and compliance with regulatory and procedural frameworks described in this specification solve such problems by allowing staff to utilize their mobile devices to view secured data and information relevant to providing patient care in the hospital and communicate with department colleagues or healthcare team members or managers. In addition to the convenience of being able to communicate to staff mobile devices, time is no longer an issue to contend with since the system would be easily accessible to staff at times that are most convenient to them.

Embodiments of the system and process for facilitating hospital staff communication, development, and compliance with regulatory and procedural frameworks described in this specification differ from and improve upon currently existing options. In particular, current methods of sharing information include staff meetings and postings on communication bulletin boards. In contrast, the system and process for facilitating hospital staff communication, development, and compliance would provide an electronic means that is available to staff wherever they are and whenever it is convenient to them.

In addition, these embodiments improve upon the currently existing options because current methods for communicating key department information is hit-and-miss, with typically only 80% of the staff viewing or hearing the information, at best. Yet, the system and process described in this specification provides access to communications at the convenience of the staff who need to receive those communications. Furthermore, the system and the process of this disclosure provides access to required education modules on clinical subjects which staff variously are assigned or need to complete.

The system and process for facilitating hospital staff communication, development, and compliance with regulatory and procedural frameworks of the present disclosure may be comprised of the following elements. This list of possible constituent elements is intended to be exemplary only and it is not intended that this list be used to limit the system and process of the present application to just these elements. Persons having ordinary skill in the art relevant to the present disclosure may understand there to be equivalent elements that may be substituted within the present disclosure without changing the essential function or operation of the system and process for facilitating hospital staff communication, development, and compliance with regulatory and procedural frameworks.

1. Manager uploads information to and posts in the mobile app for viewing by staff

2. Staff members download the mobile app onto their personal mobile device

3. System provides staff secured access to the information posted in the mobile app

4. Staff members assigned within the mobile app to view information from their assigned department

5. Staff members read and review posted information

6. Staff members perform assigned education modules as needed

7. Staff members take mandatory tests as needed

8. Staff members acknowledge review of the information provided

9. Staff members leave messages or questions in secured area accessible only to the department manager

The various elements of the system and process for facilitating hospital staff communication, development, and compliance with regulatory and procedural frameworks of the present disclosure may be related in the following exemplary fashion. It is not intended to limit the scope or nature of the relationships between the various elements and the following examples are presented as illustrative examples only. Step one is required for completion of steps five-nine. Staff can only view information if it is posted by the manager. Steps two-four allow for the assurance that staff are receiving the appropriate information relevant to them in steps five-nine.

The system and process for facilitating hospital staff communication, development, and compliance with regulatory and procedural frameworks of the present disclosure generally works so that staff can only receive the information if a manager provides the information. Staff may then access and review the information for the program to achieve any of several departmental or team unit goals, such as the goal of dissemination of pertinent information to employees of clinical departments in hospitals.

To make the system and process for facilitating hospital staff communication, development, and compliance with regulatory and procedural frameworks of the present disclosure, a person may develop an application that provides features described above in which would be compatible with mobile devices across a variety of platforms (e.g., operating systems, such as Android, iOS, etc.). As there are privacy, regulatory, and other matters of concern involved, the application may be designed to have the ability to provide information on a secured format to ensure that information in the app could not be shared or downloaded onto another device. On the other hand, the application may be designed to allow for ease of posting information.

Several more detailed embodiments are described in the sections below. Section I describes a process for facilitating hospital staff communication, development, and compliance with regulatory and procedural frameworks by reference to several block diagram and flow chart illustrations. Section II describes a network architecture of a cloud-based regulatory and procedural framework compliance and hospital staff communication and development system. Section III describes an electronic system that implements embodiments of the processes for facilitating hospital staff communication, development, and compliance with regulatory and procedural frameworks.

I. Processes for Facilitating Hospital Staff Communication, Development, and Compliance with Regulatory and Procedural Frameworks

In some embodiments, the process for facilitating hospital staff communication, development, and compliance with regulatory and procedural frameworks includes (i) receiving, from a manager computing device, information provided by a manager and related to a hospital department for viewing by staff associated with the hospital department, (ii) posting the information related to the hospital department on a newsfeed for the hospital department that is accessible to the manager via the manager computing device and the staff via a mobile app that runs on a personal mobile device for each staff member of the staff, (iii) receiving, from a first mobile device of a first staff member, a set of user credentials for a first staff member login, (iv) authenticating the set of user credentials for the first staff member login, (v) providing, to the first mobile device of the first staff member, secured access to the newsfeed when the set of user credentials for the first staff member login are successfully authenticated, and (vi) transmitting the posted information related to the hospital department to the first mobile device for display on a screen of the first mobile device when the first mobile device securely accesses the newsfeed and the first staff member selects a newsfeed entry in the newsfeed associated with the posted information.

In some embodiments, the process for facilitating hospital staff communication, development, and compliance with regulatory and procedural frameworks further comprises receiving an acknowledgment from the first mobile device that the first staff member has viewed the posted information after the posted information is displayed on the screen of the first mobile device.

In some embodiments, the posted information related to the hospital department comprises an education module for staff to complete. In some embodiments, transmitting the posted information related to the hospital department to the first mobile device comprises transmitting the education module to the first mobile device. In some embodiments, the process for facilitating hospital staff communication, development, and compliance with regulatory and procedural frameworks further comprises receiving a notification of completion from the first mobile device when the first staff member has viewed and completed the education module on the first mobile device.

In some embodiments, the posted information related to the hospital department comprises a mandatory test for staff to complete. In some embodiments, transmitting the posted information related to the hospital department to the first mobile device comprises transmitting the mandatory test to the first mobile device. In some embodiments, the process for facilitating hospital staff communication, development, and compliance with regulatory and procedural frameworks further comprises receiving a notification of mandatory test completion from the first mobile device when the first staff member has completed the mandatory test on the first mobile device.

In some embodiments, the process for facilitating hospital staff communication, development, and compliance with regulatory and procedural frameworks further comprises receiving staff information comprising at least one of a message and a question from a second mobile device associated with a second staff member, wherein the staff information is received for posting on the newsfeed. In some embodiments, the process for facilitating hospital staff communication, development, and compliance with regulatory and procedural frameworks further comprises posting the staff information in a secured area of the newsfeed that is only accessible to the manager.

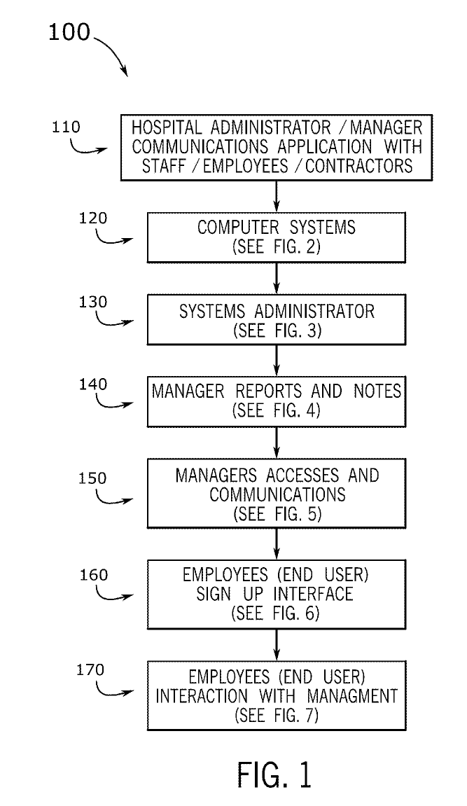

The process for facilitating hospital staff communication, development, and compliance with regulatory and procedural frameworks is now described in detail by reference to several example block diagrams and flow charts as illustrated in FIGS. 1-7.

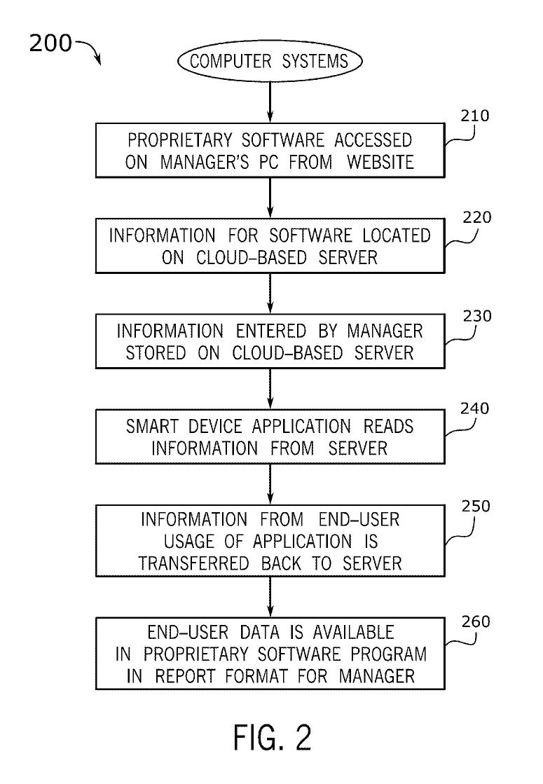

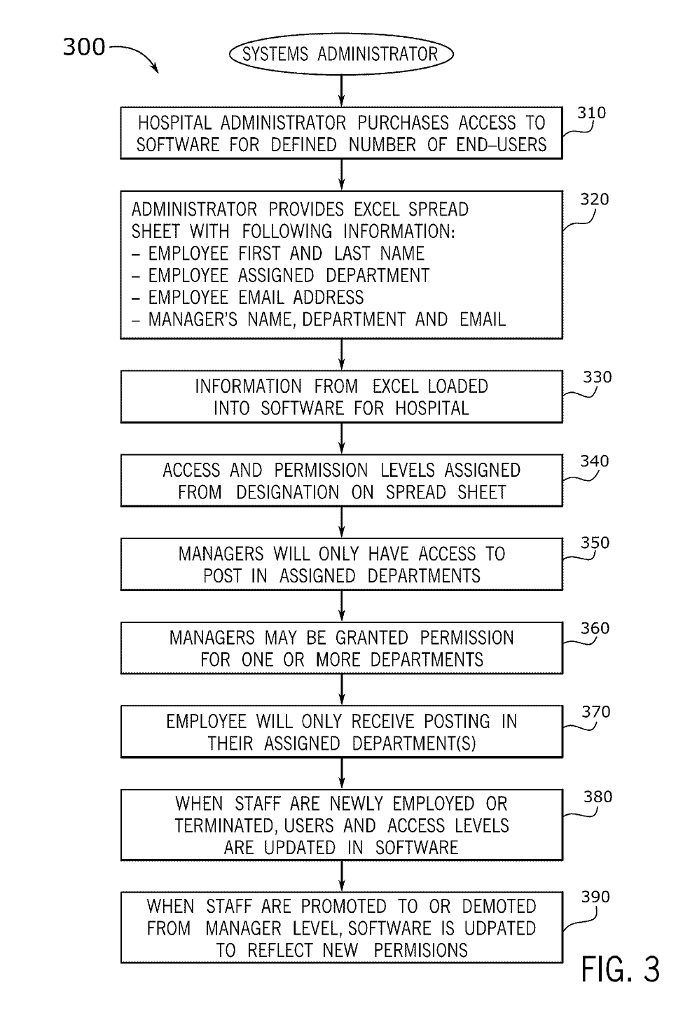

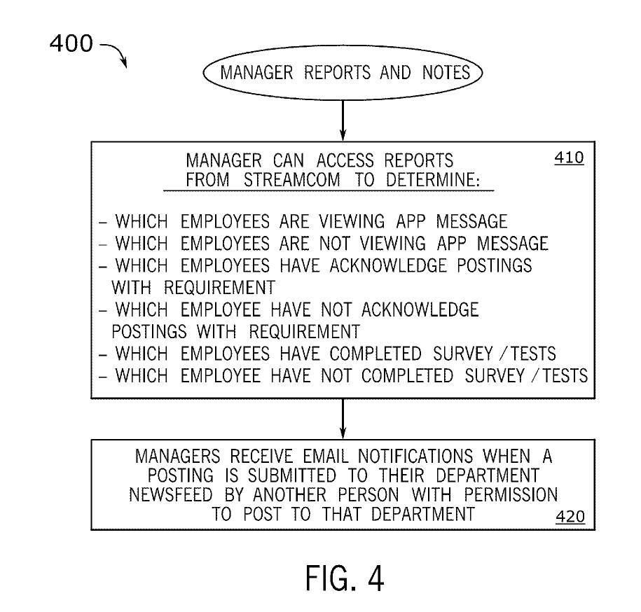

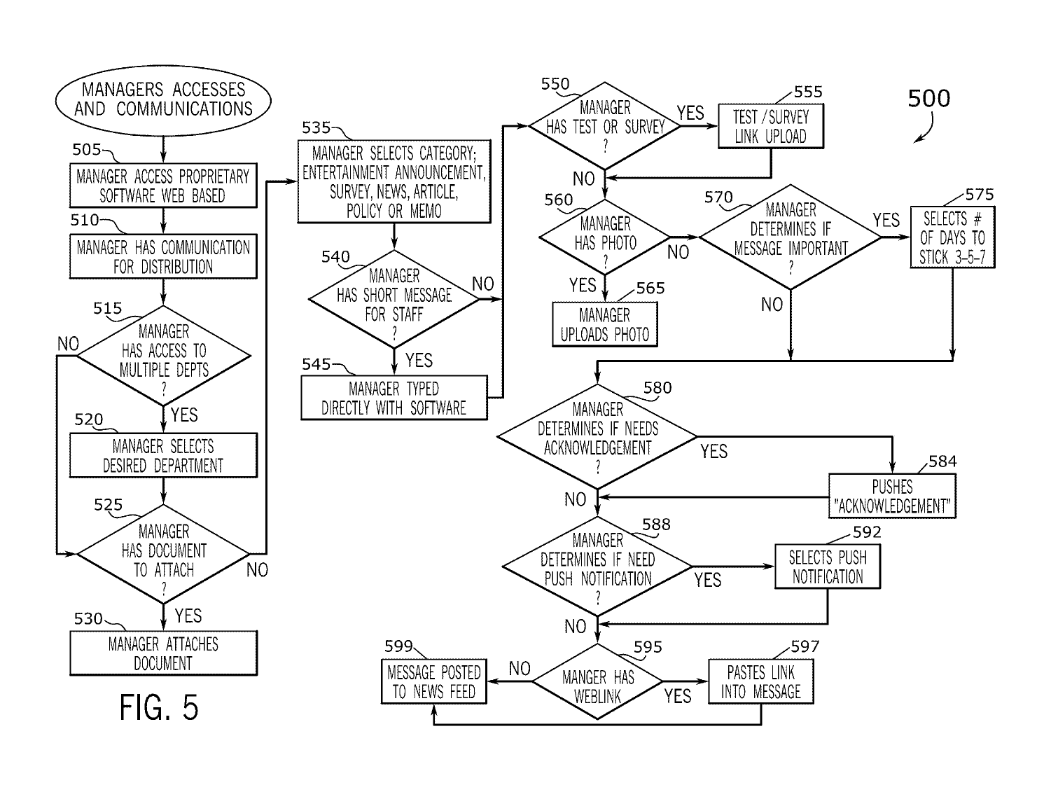

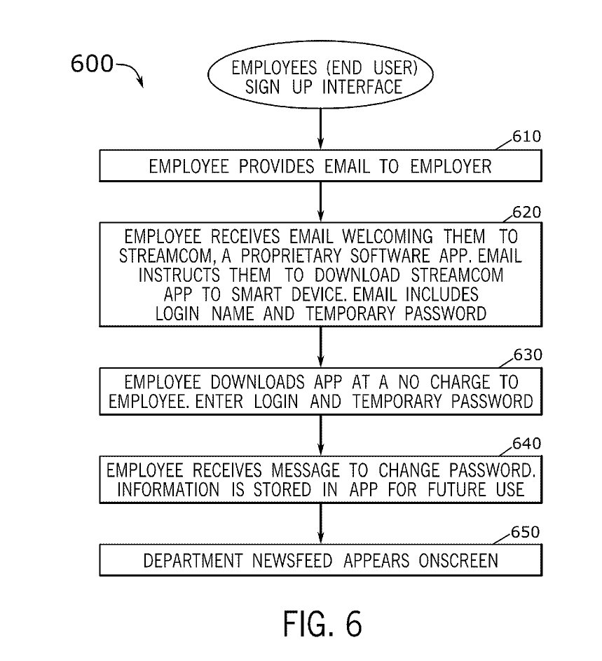

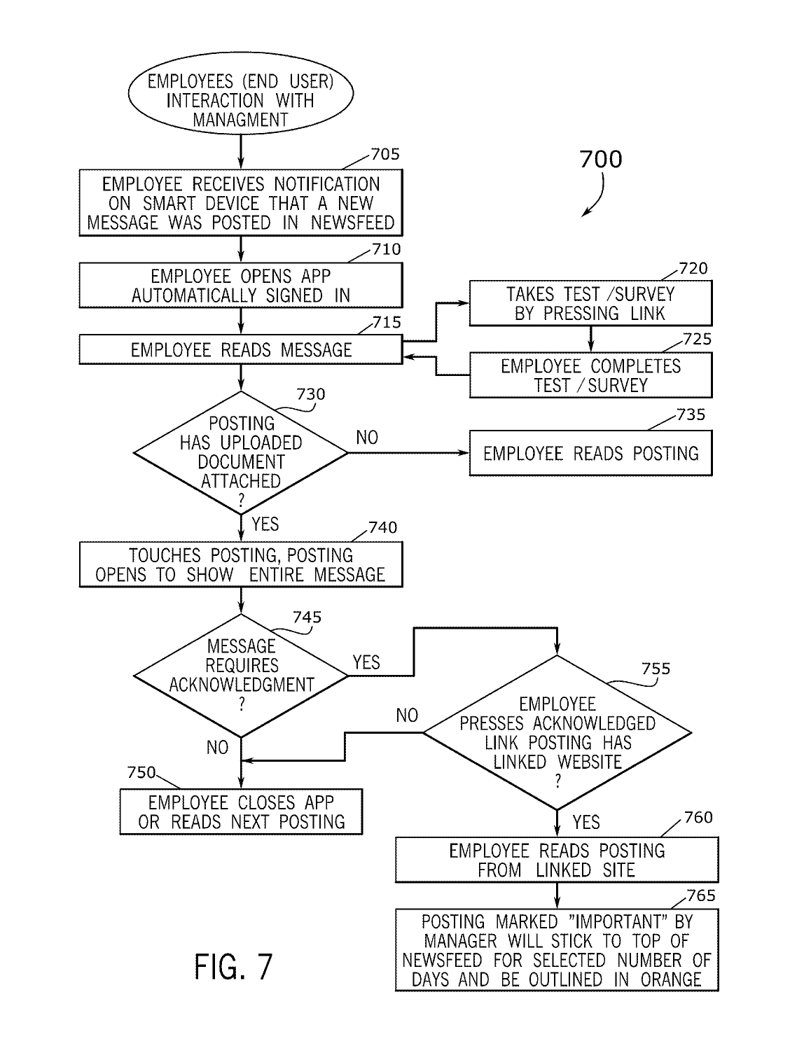

By way of example, FIG. 1 conceptually illustrates a block diagram of a mobile messaging board app for healthcare employees 100. As shown in this figure, the block diagram of the mobile messaging board app for healthcare employees 100 includes several blocks of grouped operations, machines, personnel, and actions, namely, a first block 110, a second block 120, a third block 130, a fourth block 140, a fifth block 150, a sixth block 160, and a seventh block 170. The blocks 110-170 include information about personnel, tools, and actions that are performed in furtherance of facilitating hospital staff communication, development, and compliance with regulatory and procedural frameworks. In describing the several blocks 110-170 of the block diagram of the mobile messaging board app for healthcare employees 100, reference is made to other figures. Specifically, the description of the second block 120 makes reference to FIG. 2, the description of the third block 130 makes reference to FIG. 3, the description of the fourth block 140 makes reference to FIG. 4, the description of the fifth block 150 makes reference to FIG. 5, the description of the sixth block 160 makes reference to FIG. 6, and the description of the seventh block 170 makes reference to FIG. 7.

Turning first to FIG. 1, the block diagram of the mobile messaging board app for healthcare employees 100 starts with the first block 110 which lays out the key personnel involved in facilitating hospital staff communication, development, and compliance with regulatory and procedural frameworks. In particular, the first block 110 refers to key personnel including a hospital administrator, a manager, staff, employees, and contractors. In addition, the first block 110 refers to manager communications with staff, employees, and contractors. The manager communications in this example facilitated by an application, namely, the mobile messaging board app for healthcare employees.

Next, the block diagram of the mobile messaging board app for healthcare employees 100 proceeds to the second block 120 which makes reference to FIG. 2 and indicates computer systems used in connection with carrying out the operations for facilitating hospital staff communication, development, and compliance with regulatory and procedural frameworks.

In reference to FIG. 2, the computer systems noted in the second block 120 are used in connection with carrying out the operations for facilitating hospital staff communication, development, and compliance with regulatory and procedural frameworks by several steps 210-260 of a process for facilitating hospital staff communication, development, and compliance with regulatory and procedural frameworks by way of computer systems 200. As shown in FIG. 2, the process for facilitating hospital staff communication, development, and compliance with regulatory and procedural frameworks by way of computer systems 200 starts when proprietary software is accessed (at 210) on a website through manager's computer (or “PC”). In some embodiments, the proprietary software includes the mobile messaging board app for healthcare employees. In some embodiments, the manager uploads the proprietary software when accessing the website. In this way, the manager makes the proprietary software available for staff, employees, and contractors in order to facilitate communication, development, and compliance with regulatory and procedural frameworks.

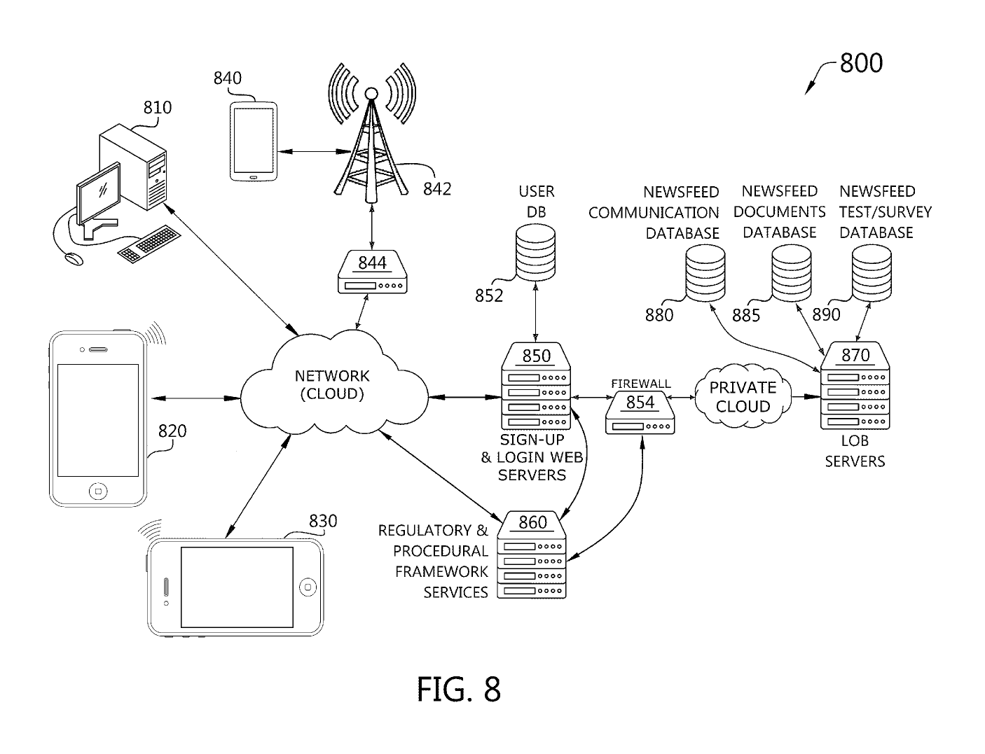

Next, the process for facilitating hospital staff communication, development, and compliance with regulatory and procedural frameworks by way of computer systems 200 indicates (at 220) that information related to the proprietary software is located on a cloud-based server. In some embodiments, the cloud-based server is a component of a cloud-based regulatory and procedural framework compliance and hospital staff communication and development system, which is further described below by reference to FIG. 8. In some embodiments, the process for facilitating hospital staff communication, development, and compliance with regulatory and procedural frameworks by way of computer systems 200 then indicates (at 230) that information entered or input by the manager is stored in the cloud in connection with the cloud-based server. In some embodiments, the information is stored in a cloud database that is communicably connected to the cloud-based server.

In some embodiments, the process for facilitating hospital staff communication, development, and compliance with regulatory and procedural frameworks by way of computer systems 200 indicates (at 240) that a smart device application reads information from the cloud-based server. For example, the mobile messaging board app for healthcare employees may be installed on a healthcare employee's mobile device (or smartphone) and the healthcare employee is using the device to access the cloud-based server to retrieve healthcare information in connection with communication between other employees, staff, contractors, or the manager. Next, the process for facilitating hospital staff communication, development, and compliance with regulatory and procedural frameworks by way of computer systems 200 indicates (at 250) that information from the end-user (e.g., the healthcare employee) is transferred back to the cloud-based server for storage in the cloud database. Along with the information, software application usage data that is tracked while the end-user uses the software is uploaded to the cloud-based server for storage in the cloud database.

In some embodiments, the process for facilitating hospital staff communication, development, and compliance with regulatory and procedural frameworks by way of computer systems 200 indicates (at 260) that end-user data is available in report format from the proprietary software for managers or system administrators (or other authorized users). In this way, all substantive communications and information is stored in connection with the cloud-based server for other employees, staff, contractors, or the manager to view depending on their respective level of privileges to read or view such information. In addition, basic usage data is tracked and stored to allow managers and system administrators to evaluate the effectiveness of the software while also tracking for compliance with regulatory frameworks or other rules.

Turning back to FIG. 1, the block diagram of the mobile messaging board app for healthcare employees 100 proceeds to the third block 130 which makes reference to FIG. 3 and indicates systems administrator involved in carrying out the operations for facilitating hospital staff communication, development, and compliance with regulatory and procedural frameworks.

Now in reference to FIG. 3, the systems administrator process for facilitating hospital staff communication, development, and compliance with regulatory and procedural frameworks 300 indicates (at 310) that a hospital administrator purchases access to software for a defined number of end users. For example, there may be ten healthcare employees on a healthcare team who need access to the mobile messaging board app, or several teams of healthcare employees, staff, contractors, etc., throughout an entire hospital may need access, which the hospital administrator can purchase and distribute. In some embodiments, the systems administrator process for facilitating hospital staff communication, development, and compliance with regulatory and procedural frameworks 300 proceeds to the next step in which the administrator provides a spreadsheet to the healthcare employees, staff, contractors, etc., with access (at 320). In some embodiments, the spreadsheet includes information for each employee (including staff and contractors) and manager. The employee and manager spreadsheet information includes at least each employee's first and last name, each employee's assigned department, each employee's email address, access and permission level of each employee, and the manager's name (first and last name), department, and email address. Next, the information from the spreadsheet is loaded (at 330) into the software for the hospital or team.

In some embodiments, the systems administrator process for facilitating hospital staff communication, development, and compliance with regulatory and procedural frameworks 300 assigns (at 340) access and permission levels from those access/permission levels designated in the spreadsheet. Additionally, the manager associated with the spreadsheet is only allowed access to post in assigned departments, as indicated in the spreadsheet (at 350). In some embodiments, managers are granted permission (at 360) to one or more departments. For instance, some managers can be granted permission for one and only one department, while some other managers can be granted permission for multiple departments.

In some embodiments, the systems administrator process for facilitating hospital staff communication, development, and compliance with regulatory and procedural frameworks 300 allows employees to receive (at 370) postings from their assigned department(s). In some embodiments, the systems administrator process for facilitating hospital staff communication, development, and compliance with regulatory and procedural frameworks 300 update user access and permission levels (at 380) when there is a change in worker status. For example, staff are hired and are newly employed, which triggers new access and permission levels to be created for the new staff, employee, contractor, etc. Alternatively, when an employee or staff member is terminated, or a contract of a contractor expires, then the access and permission levels may be revoked in relation to the contract (or new updated access rights and permission levels can be associated with the contractor when the scope and nature of work of the contractor has changed for the hospital). The software receives all such access and permission levels (new, changed, and/or revoked), to keep the software up to date. Furthermore, when staff or employees are promoted to or demoted from manager level, then the systems administrator process for facilitating hospital staff communication, development, and compliance with regulatory and procedural frameworks 300 updates (at 390) the software to reflect the change in access and permissions from employee to manager or manager to employee.

Again referring back to FIG. 1, the block diagram of the mobile messaging board app for healthcare employees 100 proceeds to the fourth block 140 which makes reference to FIG. 4 and indicates manager reports and notes that are involved in carrying out the operations for facilitating hospital staff communication, development, and compliance with regulatory and procedural frameworks.

By way of example, FIG. 4 conceptually illustrates a manager reports and notes process for facilitating hospital staff communication, development, and compliance with regulatory and procedural frameworks 400. As shown in this figure, the manager reports and notes process for facilitating hospital staff communication, development, and compliance with regulatory and procedural frameworks 400 indicates (at 410) that the manager can access reports from a system to determine status, in realtime, of several items, including, without limitation, which employees are viewing app message(s), which employees are not viewing app message(s), which employees have acknowledged postings with a requirement, which employees have not acknowledged postings with a requirement, which employees have completed a survey, a test, or another item provided by the manager for completion, and which employees have not completed the survey, the test, or other item provided by the manager for completion.

In some embodiments, the manager reports and notes process for facilitating hospital staff communication, development, and compliance with regulatory and procedural frameworks 400 next indicates (at 420) that managers receive email notifications when a posting is submitted to their department (via newsfeed, stream, or other manner) by another person with permission to post to the department.

Now returning to FIG. 1 again, the block diagram of the mobile messaging board app for healthcare employees 100 proceeds to the fifth block 150 which makes reference to FIG. 5 and cites the managers accesses and communications involved in facilitating hospital staff communication, development, and compliance with regulatory and procedural frameworks.

By way of example, FIG. 5 conceptually illustrates a manager accesses and communications process for facilitating hospital staff communication, development, and compliance with regulatory and procedural frameworks 500. As shown in this figure, the manager accesses and communications process for facilitating hospital staff communication, development, and compliance with regulatory and procedural frameworks 500 starts when a manager accesses (at 505) the proprietary software via the web. Next, the manager accesses and communications process for facilitating hospital staff communication, development, and compliance with regulatory and procedural frameworks 500 continues to the next step (at 510) in which the manager has communication(s), message(s), information, tasks, etc., for distribution to the healthcare employees, staff, and/or contractors associated with the department, the team, and/or the hospital, clinic, or healthcare facility. To complete that aforementioned step, the manager accesses and communications process for facilitating hospital staff communication, development, and compliance with regulatory and procedural frameworks 500 determines (at 515) whether the manager has access to multiple departments or only to a single department. In some embodiments, when the manager has access only to a single department, then the manager accesses and communications process for facilitating hospital staff communication, development, and compliance with regulatory and procedural frameworks 500 skips ahead to determine (at 525) whether the manager has a document to attach, which is further described below. On the other hand, when the manager has access to multiple departments, then the manager accesses and communications process for facilitating hospital staff communication, development, and compliance with regulatory and procedural frameworks 500 proceeds to a step at which the manager selects (at 520) the department to work with in this case.

In some embodiments, the manager accesses and communications process for facilitating hospital staff communication, development, and compliance with regulatory and procedural frameworks 500 determines (at 525) whether the manager has a document to attach. When the manager has a document to attach, then the manager accesses and communications process for facilitating hospital staff communication, development, and compliance with regulatory and procedural frameworks 500 proceeds to a step at which the manager attaches the document (at 530). On the other hand, when there is no document to attach (or after attaching the document), the manager accesses and communications process for facilitating hospital staff communication, development, and compliance with regulatory and procedural frameworks 500 continues to the next step at which the manager selects a category (at 535). In some embodiments, the categories from which the manager can select include, without limitation, entertainment announcement, survey, news, article, policy, or memo. In some embodiments, additional categories are included, depending on the nature of the department or team needs.

In some embodiments, the manager accesses and communications process for facilitating hospital staff communication, development, and compliance with regulatory and procedural frameworks 500 determines (at 540) whether the manager has a short message for staff, employees, and/or contractors in the department or team. When the manager has a short message, then the manager accesses and communications process for facilitating hospital staff communication, development, and compliance with regulatory and procedural frameworks 500 allows the manager to type (at 545) the short message directly with the software. Then the manager accesses and communications process for facilitating hospital staff communication, development, and compliance with regulatory and procedural frameworks 500 proceeds to the next step to determine (at 550) whether the manager has a test or survey.

Turning back to the determination (at 540), when the manager does not have a short message to share with the staff, employees, or contractors, then the manager accesses and communications process for facilitating hospital staff communication, development, and compliance with regulatory and procedural frameworks 500 determines (at 550) whether the manager has a test or survey. When the manager has a test or survey, then the manager accesses and communications process for facilitating hospital staff communication, development, and compliance with regulatory and procedural frameworks 500 uploads (at 555) the test/survey to provide a link for the staff, employees, and/or contractors to access the test/survey. Then the manager accesses and communications process for facilitating hospital staff communication, development, and compliance with regulatory and procedural frameworks 500 proceeds to the next step to determine (at 560) whether the manager has a photo to upload.

Turning back to the determination (at 550), when the manager does not have a test/survey to upload, then the manager accesses and communications process for facilitating hospital staff communication, development, and compliance with regulatory and procedural frameworks 500 determines (at 560) whether the manager has a photo to upload. When the manager has a photo, then the manager accesses and communications process for facilitating hospital staff communication, development, and compliance with regulatory and procedural frameworks 500 uploads (at 565) the photo. On the other hand, when the manager does not have a photo to upload, then the manager accesses and communications process for facilitating hospital staff communication, development, and compliance with regulatory and procedural frameworks 500 determines (at 570) whether the manager thinks the message is important. When the message is important, then the manager accesses and communications process for facilitating hospital staff communication, development, and compliance with regulatory and procedural frameworks 500 selects (at 575) a number of days to stick the message (e.g., three, five, seven, etc., days), and then proceeds to the next step (at 580).

However, when the message is not important, then the manager accesses and communications process for facilitating hospital staff communication, development, and compliance with regulatory and procedural frameworks 500 determines (at 580) whether the message needs to be acknowledged by staff, employees, or contractors. When acknowledgment is needed, then the manager accesses and communications process for facilitating hospital staff communication, development, and compliance with regulatory and procedural frameworks 500 pushes (at 584) the “acknowledgment” to staff, employees, and contractors who are to view the message, and then proceeds to the next step (at 588).

On the other hand, when acknowledgment is not needed, then the manager accesses and communications process for facilitating hospital staff communication, development, and compliance with regulatory and procedural frameworks 500 determines (at 588) whether push notification is required. When push notification is required, then the manager selects (at 592) push notification and proceeds to the next step (at 595). However, when push notification is not required, then the manager accesses and communications process for facilitating hospital staff communication, development, and compliance with regulatory and procedural frameworks 500 determines (at 595) whether the manager has a web link to share in a message. When the manager has a web link, then the manager accesses and communications process for facilitating hospital staff communication, development, and compliance with regulatory and procedural frameworks 500 pastes (at 597) the web link into the message and proceeds to post (at 599) the message (with or without the web link) to a new feed for the staff, employees, contractors, etc.

Referring back to FIG. 1, the block diagram of the mobile messaging board app for healthcare employees 100 proceeds to the sixth block 160 which makes reference to FIG. 6 and notes the employee (end user) sign up interface accessed by employees, staff, and/or contractors to use the software for facilitating hospital staff communication, development, and compliance with regulatory and procedural frameworks.

By way of example, FIG. 6 conceptually illustrates an employee (end user) sign up interface process 600. As shown in this figure, the employee (end user) sign up interface process 600 starts when an employee provides (at 610) email to an employer. In some embodiments, the employee then receives (at 620) email welcoming them to the streaming system, as a proprietary software application. In some embodiments, the email instructs the employee to download the software application to a device (e.g., download mobile app to mobile device). In some embodiments, the email also includes a login name and temporary password, which the user can change.

In some embodiments, the employee (end user) sign up interface process 600 proceeds to the next step at which the employee downloads (at 630) the software app (e.g., at no charge to the employee), and enters the login username and temporary password. In some embodiments, the employee (end user) sign up interface process 600 proceeds to the next step at which the employee receives (at 640) a message to change the temporary password. Information is then stored in the app for future use. Once all the above steps are completed, the employee (end user) sign up interface process 600 displays (at 650) the department or team news feed on the screen of the user's device.

Turning back to FIG. 1, the block diagram of the mobile messaging board app for healthcare employees 100 proceeds to the seventh block 170 which makes reference to FIG. 7 and describes employee (end user) interactions with management that occur while carrying out the operations for facilitating hospital staff communication, development, and compliance with regulatory and procedural frameworks.

Now turning to another example, FIG. 7 conceptually illustrates an employee (end user) interactions with management process 700. As shown in this figure, the employee (end user) interactions with management process 700 starts when employee receives (at 705) a notification on the employee's device (e.g., smartphone), with the notification indicating that a message was posted in the news feed for the department or team to which the employee is a member. Next, the employee opens (at 710) the software app on the device. The software app may be automatically configured to login with saved username/password. Alternatively, the employee may sign up by entering username and password. In some embodiments, the employee then reads (at 715) the message that was posted per the notification. When the message posted in the news feed includes a test or survey, then the employee takes (at 720) the posted test/survey by pressing a link to access the test/survey. Next, the employee completes the test/survey (at 725).

In some embodiments, the employee (end user) interactions with management process 700 then determines (at 730) whether the posted message includes an attached document or not. When there is no document attached to the posted message, the employee (end user) interactions with management process 700 of some embodiments allows for the employee to then read the posting (at 735). On the other hand, when there is a document attached to the posted message, then the employee opens the posted message (at 740) to show the entire message and attached document. For example, the employee may touch the screen of the mobile device to open the message and attached document.

In some embodiments, the employee (end user) interactions with management process 700 then determines (at 745) whether the message requires an acknowledgment. When acknowledgment is not required, then the employee (end user) interactions with management process 700 proceeds to the employee closing (at 750) the app or reading the next posted message (if any). However, when acknowledgment is required, then the employee (end user) interactions with management process 700 determines (at 755) whether the employee has selected an acknowledgment link posting that links to the website. When the employee has selected the acknowledgment link, then the employee (end user) interactions with management process 700 continues for the employee to read (at 760) a posting from the linked website, and then to sticking (at 765) the posting marked “important” by the manager to the top of the news feed for a selected number of days and highlighting for visibility. Then the employee (end user) interactions with management process 700 proceeds to allow the employee to close the app (at 750) or read the next posting in the news feed.

### II. Cloud-Based Regulatory and Procedural Framework Compliance and Hospital Staff Communication and Development System

By way of example, FIG. 8 conceptually illustrates a network architecture of a cloud-based regulatory and procedural framework compliance and hospital staff communication and development system 800.

As shown in this figure, the cloud-based regulatory and procedural framework compliance and hospital staff communication and development system 800 includes several client-side computing devices, including a desktop computer 810, a first mobile computing device 820, a second mobile computing device 830, and a tablet computing device 840. The cloud-based regulatory and procedural framework compliance and hospital staff communication and development system 800 also includes a wireless communication point 842 (e.g., a cell tower for cellular data communication), a gateway 844, a set of front end cloud server computing devices 850 (for sign up and login by employees, staff, contractors, managers, and other healthcare users of the client-side computing devices when connected to the web portal on the website), a healthcare user database 852, a firewall 854, servers of third party healthcare regulatory and procedural framework services 860, a set of regulatory and procedural framework compliance and hospital staff communication servers 870, a newsfeed communication database 880, a newsfeed documents database 885, and a newsfeed test/survey database 890.

The client-side computing devices connect to the front end cloud server computing devices 850 over a network (“cloud”), such as the Internet, in order to sign up or login to the cloud-based regulatory and procedural framework compliance and hospital staff communication and development system 800. In some embodiments, when a client-side computing device first connects to the front end cloud server computing devices 850, the client-side computing device is provided a link to download a software application that offers a full set of features for interacting with the cloud-based regulatory and procedural framework compliance and hospital staff communication and development system 800. After sign up and login, the client-side computing devices each open a session over a private cloud network to the set of regulatory and procedural framework compliance and hospital staff communication servers 870. Each of the client-side computing devices includes a graphical user interface (GUI)-based software application or GUI-based mobile app. The GUIs of the software application and the mobile app allow healthcare staff, employees, contractors, managers, administrators, and other healthcare users to navigate the features of the cloud-based regulatory and procedural framework compliance and hospital staff communication and development system 800.

Indirect connection is also supported. Specifically, the tablet computing device 840 connects to the wireless communication point 842 to connect (via the gateway 844) to the front end cloud server computing devices 850, which process connection requests from the client-side computing devices. Typically, the connection requests come with basic user information for sign up or, when already signed up, with user credentials for login. The login user credentials are checked for validity by a user authentication module of the front end cloud server computing devices 850 based on encrypted user credentials stored in the healthcare user database 852. The healthcare user database 852 also includes information pertaining to department assignments per manager and department teams associated with the staff, employees, contractors, etc. The encrypted user credentials and departmental information are stored in the healthcare user database 852 after sign up for each healthcare staff member, employee, contractor, and manager, as specified by invite, and authorized by permission levels, from one or more administrators of the cloud-based regulatory and procedural framework compliance and hospital staff communication and development system 800.

Upon valid authentication of the login user credentials, a session is started in relation to the client-side computing device of the healthcare end user (e.g., staff member, employee, contractor, etc.), thereby allowing the healthcare user to access the set of regulatory and procedural framework compliance and hospital staff communication servers 870 over the private cloud network.

Also, the regulatory and procedural framework compliance and hospital staff communication servers 870 are line of business servers (LOB server computing devices) with at least a server-side newsfeed software application and a database management system for storing data in and retrieving data from the newsfeed communication database 880, the newsfeed documents database 885, and the newsfeed test/survey database 890. The regulatory and procedural framework compliance and hospital staff communication servers 870, the newsfeed communication database 880, the newsfeed documents database 885, and the newsfeed test/survey database 890 are accessed over the private network connection for the client-side computing device session through a firewall 854 that secures data at HIPPA compliance standards or higher and provides system-wide data integrity for the regulatory and procedural framework compliance and hospital staff communication servers 870, the newsfeed communication database 880, the newsfeed documents database 885, and the newsfeed test/survey database 890.

### III. Electronic System

Many of the above-described features and applications are implemented as software processes that are specified as a set of instructions recorded on a computer readable storage medium (also referred to as computer readable medium or machine readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

In this specification, the term “software” is meant to include firmware residing in read-only memory or applications stored in magnetic storage, which can be read into memory for processing by a processor. Also, in some embodiments, multiple software inventions can be implemented as sub-parts of a larger program while remaining distinct software inventions. In some embodiments, multiple software inventions can also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

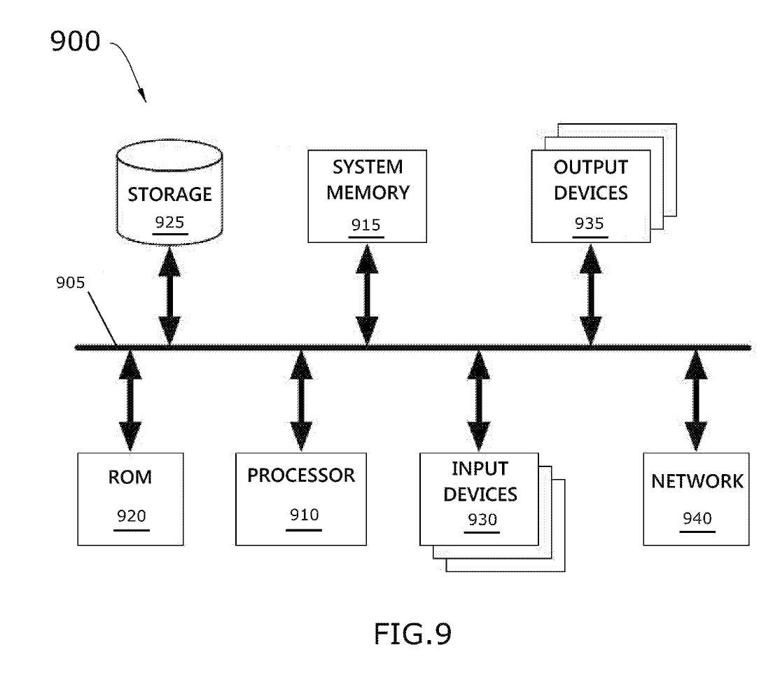

FIG. 9 conceptually illustrates an electronic system 900 with which some embodiments of the invention are implemented. The electronic system 900 may be a computer, phone, PDA, or any other sort of electronic device. Such an electronic system includes various types of computer readable media and interfaces for various other types of computer readable media. Electronic system 900 includes a bus 905, processing unit(s) 910, a system memory 915, a read-only 920, a permanent storage device 925, input devices 930, output devices 935, and a network 940.

The bus 905 collectively represents all system, peripheral, and chipset buses that communicatively connect the numerous internal devices of the electronic system 900. For instance, the bus 905 communicatively connects the processing unit(s) 910 with the read-only 920, the system memory 915, and the permanent storage device 925.

From these various memory units, the processing unit(s) 910 retrieves instructions to execute and data to process in order to execute the processes of the invention. The processing unit(s) may be a single processor or a multi-core processor in different embodiments.

The read-only-memory (ROM) 920 stores static data and instructions that are needed by the processing unit(s) 910 and other modules of the electronic system. The permanent storage device 925, on the other hand, is a read-and-write memory device. This device is a non-volatile memory unit that stores instructions and data even when the electronic system 900 is off. Some embodiments of the invention use a mass-storage device (such as a magnetic or optical disk and its corresponding disk drive) as the permanent storage device 925.

Other embodiments use a removable storage device (such as a floppy disk or a flash drive) as the permanent storage device 925. Like the permanent storage device 925, the system memory 915 is a read-and-write memory device. However, unlike storage device 925, the system memory 915 is a volatile read-and-write memory, such as a random access memory. The system memory 915 stores some of the instructions and data that the processor needs at runtime. In some embodiments, the invention's processes are stored in the system memory 915, the permanent storage device 925, and/or the read-only 920. For example, the various memory units include instructions for processing appearance alterations of displayable characters in accordance with some embodiments. From these various memory units, the processing unit(s) 910 retrieves instructions to execute and data to process in order to execute the processes of some embodiments.

The bus 905 also connects to the input and output devices 930 and 935. The input devices enable the user to communicate information and select commands to the electronic system. The input devices 930 include alphanumeric keyboards and pointing or cursor control devices. The output devices 935 display images generated by the electronic system 900. The output devices 935 include printers and display devices, such as cathode ray tubes (CRT) or liquid crystal displays (LCD). Some embodiments include a touchscreen that functions as both an input and output device.

Finally, as shown in FIG. 9, bus 905 also couples electronic system 900 to a network 940 through a network adapter (not shown). In this manner, the computer can be a part of a network of computers (such as a local area network (“LAN”), a wide area network (“WAN”), or an Intranet), or a network of networks (such as the Internet). Any or all components of electronic system 900 may be used in conjunction with the invention.

These functions described above can be implemented in digital electronic circuitry, in computer software, firmware or hardware. The techniques can be implemented using one or more computer program products. Programmable processors and computers can be packaged or included in mobile devices. The processes and logic flows may be performed by one or more programmable processors and by sets of programmable logic circuitry. General and special purpose computing and storage devices can be interconnected through communication networks.

Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the invention has been described with reference to numerous specific details, one of ordinary skill in the art will recognize that the invention can be embodied in other specific forms without departing from the spirit of the invention. For instance, FIGS. 1-7 conceptually illustrate processes. The specific operations of each process may not be performed in the exact order shown and described. Specific operations may not be performed in one continuous series of operations, and different specific operations may be performed in different embodiments. Furthermore, each process could be implemented using several sub-processes, or as part of a larger macro process. Thus, one of ordinary skill in the art would understand that the invention is not to be limited by the foregoing illustrative details, but rather is to be defined by the appended claims.

I claim:

1. A non-transitory computer readable medium storing a hospital staff communication and development program which, when executed by at least one processing unit of a computing device, facilitates hospital staff communication, development, and compliance with regulatory and procedural frameworks, said hospital staff communication and development program comprising sets of instructions for:
receiving, from a manager computing device, information provided by a manager and related to a hospital department for viewing by staff associated with the hospital department;
posting the information related to the hospital department on a newsfeed for the hospital department that is accessible to the manager via the manager computing device and the staff via a mobile app that runs on a personal mobile device for each staff member of the staff;
receiving, from a first mobile device of a first staff member, a set of user credentials for a first staff member login;
authenticating the set of user credentials for the first staff member login;
providing, to the first mobile device of the first staff member, secured access to the newsfeed when the set of user credentials for the first staff member login are successfully authenticated; and
transmitting the posted information related to the hospital department to the first mobile device for display on a screen of the first mobile device when the first mobile device securely accesses the newsfeed and the first staff member selects a newsfeed entry in the newsfeed associated with the posted information.

2. The non-transitory computer readable medium of claim 1, wherein hospital staff communication and development program further comprises a set of instructions for receiving an acknowledgment from the first mobile device that the first staff member has viewed the posted information after the posted information is displayed on the screen of the first mobile device.

3. The non-transitory computer readable medium of claim 1, wherein the posted information related to the hospital department comprises an education module for staff to complete.

4. The non-transitory computer readable medium of claim 3, wherein the set of instructions for transmitting the posted information related to the hospital department to the first mobile device comprises a set of instructions for transmitting the education module to the first mobile device.

5. The non-transitory computer readable medium of claim 4, wherein hospital staff communication and development program further comprises a set of instructions for receiving a notification of completion from the first mobile device when the first staff member has viewed and completed the education module on the first mobile device.

6. The non-transitory computer readable medium of claim 1, wherein the posted information related to the hospital department comprises a mandatory test for staff to complete.

7. The non-transitory computer readable medium of claim 6, wherein the set of instructions for transmitting the posted information related to the hospital department to the first mobile device comprises a set of instructions for transmitting the mandatory test to the first mobile device.

8. The non-transitory computer readable medium of claim 7, wherein hospital staff communication and development program further comprises a set of instructions for receiving a notification of mandatory test completion from the first mobile device when the first staff member has completed the mandatory test on the first mobile device.

9. The non-transitory computer readable medium of claim 1, wherein the hospital staff communication and development program further comprises a set of instructions for receiving staff information to post on the newsfeed from a second mobile device associated with a second staff member, wherein the information to post comprises at least one of a message and a question.

10. The non-transitory computer readable medium of claim 9, wherein the hospital staff communication and development program further comprises a set of instructions for posting the staff information in a secured area of the newsfeed that is only accessible to the manager.
